(19) United States
(12) Patent Application Publication
(10) Pub. No.: US 2022/0167694 A1
(43) Pub. Date: Jun. 2, 2022

(54) HEALTH BAND FOR HUMAN BODY

(71) Applicant: Jong Deuk Park, Seoul (KR)

(72) Inventor: Jong Deuk Park, Seoul (KR)

(21) Appl. No.: 17/442,838

(22) PCT Filed: Mar. 23, 2020
(86) PCT No.: PCT/KR2020/003974

§ 371 Date: Sep. 24, 2021

(30) Foreign Application Priority Data
Mar. 24, 2019 (KR) 20-2019-0001202
Jun. 22, 2019 (KR) 10-2019-0074662
Feb. 17, 2020 (KR) 20-2020-0000562
Feb. 22, 2020 (KR) 20-2020-0000600

(51) Int. Cl. A41D 13/11; A62B 23/00

(52) U.S. Cl. CPC A41D 13/1169; A62B 23/00; A41D 13/1107

(57) ABSTRACT

A health band for a human body may include a band part having a through hole formed at a predetermined position such that a large amount of air for breathing by the respiratory system of the human body is introduced; and a protruding member which is formed so as to surround the through hole while blocking the through hole so as to prevent substances harmful to the human body contained in the air introduced through the through hole from flowing into the respiratory system.

### BACKGROUND

As well known, masks are classified into surgical masks, healthcare masks, dust masks, cold weather masks, and general masks, and the like, and as people are very interested in their healthcare according to environments, recently, demands for healthcare masks have been drastically increased.

The healthcare masks may be categorized into antimicrobial masks, yellow dust masks, fine dust masks, and the like, and the healthcare mask is constituted of several filter layers to the shape (structure) of surrounding the mouth and nose of a human body. According to the recommendations from the World Health Organization (WHO), generally, the healthcare mask should be used as a disposable product.

The antimicrobial mask functions to prevent (filter) bacteria, fungi, viruses, and the like from being introduced into the human body through the mouth or nose, the yellow dust mask functions to prevent (filter) yellow sand frequently generated recently due to environment pollution from being introduced into the human body through the mouth or nose, the fine dust mask functions to prevent (filter) fine and ultrafine substances, such as bacteria, fungi, viruses, yellow sand, carcinogens, and the like, which are frequently generated due to environment pollution, from being introduced into the human body through the mouth or nose.

As conventional healthcare masks are configured to cover all of the mouth and nose of the human body, they are filled with large amounts of moisture and carbon dioxide generated when a wearer breathes at the insides thereof, thereby obtaining no wearing comfort and having headache and difficulties in breathing, and as bad breath remains in the masks, further, it is hard to wear the masks for long hours due to such unpleasant feelings.

Moreover, conventional surgical masks are configured to widely surround all of the mouth and nose of the human body to protect the wearer from droplet infection or air infection due to all kinds of bacteria, viruses, and harmful substances, so that they can be hard to be worn for long hours.

In the case of the user who wears glasses, also, the conventional masks make the glasses fog due to a large amount of moisture filled at the insides thereof, and as the interiors of the masks are full of carbon monoxide generated when the wearer breathes, the wearer sucks the carbon monoxide again, so that while he or she is driving a car, for example, car accidents may happen due to his or her drowsy driving. Further, he or she may have headache or difficulties in breathing, thereby making it hard to wear the masks for long hours, and as the masks are configured to cover all of the mouth and nose of the human body, besides, it is very uncomfortable if the wearer takes outdoor exercise.

Additionally, the conventional masks widely cover all of the mouth and nose of the human body, but it is impossible that they come into close contact with the wearer's face, without any gap, so that through the gap, bacteria, viruses, yellow sand, carcinogens, and harmful fine substances may be introduced into the wearer's mouth and nose.

Moreover, the conventional masks widely cover all of the mouth and nose of the human body, so that when it is hot or in summer, the insides of the masks are full of sweat and it is hard for the wearer to breathe, thereby making it hard to wear the masks anymore.

Further, the conventional masks widely cover all of the mouth and nose of the human body to prevent bacteria, viruses, yellow sand, fungi, carcinogens, and harmful fine substances from being introduced through the respiratory system of the human body, thereby causing unnecessary consumption of a material.

Also, the conventional masks widely cover all of the mouth and nose of the human body, so that they are inconvenient to be carried due to large volumes and there are limitations in providing disposable masks at low prices.

As known, conventional detection kits serve to detect cancers and diseases through feces, urine, blood, and the like, and accordingly, people go to hospitals to draw blood. Further, it is hard that the detection kits are used, while being carried, and a lot of hours are needed for detection results. Also, detection methods are difficult, and through the detection kits, normally, it is hard to check whether diseases occur or not every day.

As known, conventional medicine kits are attached to a user's skin, so that a lot of hours are required until medical effects occur through the medicines permeating the skin, and further, treatment effects thereof may become deteriorated.

As known, conventional bands (including band-aid, patch, skin, pain relieving patch) are attached to a user's skin, so that some of the bands have very fine holes through which air passes toward the skin, but it is impossible to introduce a large amount of air required for breathing into the respiratory system (nasal or oral cavity) of the wearer.

### BRIEF SUMMARY

The present invention relates to a healthcare band for a human body, and more particularly, to a healthcare band for a human body that is capable of selectively covering only the mouth or nose of the human body, not surrounding the wide range covering the oral cavity (mouth) and the nasal cavity (nose) of the human body, thereby preventing substances harmful to the human body from being introduced into the human body through the respiratory system of the human body.

It is an object of the present invention to provide a healthcare band for a human body that is capable of selectively covering only the mouth or nose of the human body, not surrounding a wide range of all of the mouth and nose.

It is another object of the present invention to provide a healthcare band for a human body that is capable of selectively covering only the mouth or nose of the human body, thereby improving the wearing comfort of a wearer.

It is yet another object of the present invention to provide a healthcare band for a human body that is capable of selectively covering only the mouth or nose of the human body, thereby preventing harmful substances from being introduced into gaps occurring after worn.

It is still another object of the present invention to provide a healthcare band for a human body that is capable of selectively covering only the mouth or nose of the human body, so that the healthcare band can be used as a disposable product at a place needed.

It is still yet another object of the present invention to provide a healthcare band for a human body that is capable of selectively covering only the mouth or nose of the human body, so that as the healthcare band is relatively small in volume, it is easy to be carried in a wearer's wallet, backpack, or pocket, as items necessary for his or her trip.

It is another object of the present invention to provide a healthcare band for a human body that is capable of selectively covering only the mouth or nose of the human body, so that the healthcare band can be used even in hot regions or in summers.

It is still another object of the present invention to provide a healthcare band for a human body that is capable of being worn on a wearer's respiratory system when he or she cooks, thereby preventing all kinds of carcinogens and fine substances harmful to the human body that are generated when he or she cooks with gas or fire, while an air cleaner is distant from a kitchen or cooking equipment to thus fail to immediately suck the carcinogens and the harmful fine substances, from being sucked to his or her respiratory system.

It is yet another object of the present invention to provide a healthcare band for a human body that is capable of being worn on a wearer's respiratory system when he or she cleans, thereby preventing fine substances and all kinds of harmful substances scattering to the air when he or she cleans, while an air cleaner is placed at a given space in the house to thus fail to immediately suck the fine substances and all kinds of harmful substances, from being sucked to his or her respiratory system.

It is still yet another object of the present invention to provide a healthcare band for a human body that is capable of selectively covering only the mouth or nose of the human body through the point adhesion member not harmful to the human body, so that since there is no gap, the harmful substances and fine substances introduced through the gap can be prevented from entering the respiratory system of the human body.

It is another object of the present invention to provide a healthcare band for a human body that is capable of selectively covering only the mouth or nose of the human body, while being provided with a sensor module adapted to in real time monitor and store biological signals including a temperature, humidity, sweat, and the like through a mobile application, based on substances discharged from the respiratory system of the human body, and to transmit the monitored result to an external receiver, thereby capable of detecting various diseases, degrees of obesity, and health conditions.

It is yet another object of the present invention to provide a healthcare band for a human body that is capable of selectively covering only the mouth or nose of the human body to allow various medicines capable of treating diseases, obesity, and the like to directly permeate the respiratory system of the human body quickly.

It is still another object of the present invention to provide a healthcare band for a human body that is capable of selectively covering only the mouth or nose of the human body, so that the healthcare band can be worn for long hours, without any trouble.

It is still yet another object of the present invention to provide a healthcare band for a human body that is capable of selectively covering only the mouth or nose of the human body, thereby preventing a wearer's glasses from fogging due to the moisture discharged from him or her breathing.

It is another object of the present invention to provide a healthcare band for a human body that is capable of selectively covering only the mouth or nose of the human body, thereby preventing the carbon monoxide emitted from the respiratory system of the human body from being sucked again to the human body through the respiratory system.

It is yet another object of the present invention to provide a healthcare band for a human body that is capable of selectively covering only the mouth or nose of the human body, thereby improving the wearing comfort of a wearer, while he or she is taking outdoor exercise or doing recreation activities such as dancing, singing, training, and the like.

It is still another object of the present invention to provide a healthcare band for a human body that is capable of selectively covering only the mouth or nose of the human body, thereby causing no unnecessary consumption of a material.

It is yet still another object of the present invention to provide a healthcare band for a human body that is capable of selectively covering only the mouth or nose of the human body, so that the healthcare band can be worn for long hours.

It is another object of the present invention to provide a healthcare band for a human body that is capable of selectively covering only the mouth or nose of the human body, thereby being provided at low prices.

The technical problems to be achieved through the present invention are not limited as mentioned above, and other technical problems not mentioned herein will be understood to one of ordinary skill in the art through the following description.

### Technical Solutions

To accomplish the above-mentioned objects, according to an aspect of the present invention, there is provided a healthcare band for a human body, including: a band part detachably attached freely to the skin of the human body to surround the region around the respiratory system of the human body through a point adhesion member formed on one side thereof and having a through hole formed at a predetermined position thereof to introduce a large amount of air into the respiratory system of the human body; and a protruding member adapted to block the through hole, while surrounding the region around the through hole, to thus prevent substances harmful to the human body contained in the air introduced through the through hole from flowing into the respiratory system.

To accomplish the above-mentioned objects, according to another aspect of the present invention, there is provided a healthcare band for a human body, including: a band part detachably attached freely to the skin of the human body to surround the region around the respiratory system of the human body through a point adhesion member formed on one side thereof and having a through hole formed at a predetermined position thereof to introduce a large amount of air into the respiratory system of the human body; and a protruding member adapted to surround the region around the through hole and having a sensor module adapted to in real time monitor and store biological signals including a temperature, humidity, sweat, germs, viruses, carcinogens, and the like through a mobile application, based on substances discharged from the respiratory system through the through hole, to thus detect various diseases, degrees of obesity, and health conditions.

To accomplish the above-mentioned objects, according to yet another aspect of the present invention, there is provided a healthcare band for a human body, including: a band part detachably attached freely to the skin of the human body to surround the region around the respiratory system of the human body through a point adhesion member formed on one side thereof and having a through hole formed at a predetermined position thereof to introduce a large amount of air into the respiratory system of the human body; and a protruding member adapted to surround the region around the through hole and having various medicines sucked to the respiratory system through the through hole to thus treat various diseases, obesity, and health problems.

According to the present invention, the protruding member may include: an outer layer for blocking relatively large particles and water; an anti-bacterial filter layer for primarily filtering first substances harmful to the human body contained in the air passing through the outer layer; an electrostatic filter layer for secondarily filtering the first substances harmful to the human body and second substances harmful to the human body; and an inner layer for guiding the air secondarily filtered through the electrostatic filter layer to the respiratory system.

According to the present invention, the first substances harmful to the human body may include at least one or more substances selected from viruses, bacteria, carcinogens, microorganisms, and pollutants.

According to the present invention, the second substances harmful to the human body may include at least one or more substances selected from yellow sand, fine dust, ultra fine dust, and fine substances.

According to the present invention, the outer layer has the shape of lips so that while a wearer is talking, his or her lips are hidden to provide a good outer appearance.

According to the present invention, the anti-bacterial filter layer may contain the substances good to the human body, such as mineral, vitamin, and the like.

According to the present invention, the anti-bacterial filter layer may protect the human body from ultraviolet rays, radioactive rays, and the like.

According to the present invention, the electrostatic filter layer may include a melt blown (MB) filter structure material made by adding an electrostatic force to a non-woven fabric produced in a melt blown process.

According to the present invention, the inner layer may have a structure capable of accommodating a medicine permeating the human body therein.

According to the present invention, the protruding member may include: an outer layer for blocking relatively large particles and water; an anti-bacterial filter layer for primarily filtering first substances harmful to the human body contained in the air passing through the outer layer; an electrostatic filter layer for secondarily filtering the first substances harmful to the human body and second substances harmful to the human body; an inner layer for guiding the air secondarily filtered through the electrostatic filter layer to the respiratory system; and a detection kit layer formed on one side of the inner layer and having a sensor module adapted to in real time monitor and store biological signals including a temperature, humidity, sweat, germs, viruses, carcinogens, and the like through a mobile application, based on substances discharged from the respiratory system, and to transmit the monitored result to an external receiver to thus detect various diseases, degrees of obesity, and health conditions.

According to the present invention, the protruding member may include: an outer layer for blocking relatively large particles and water; an anti-bacterial filter layer for primarily filtering first substances harmful to the human body contained in the air passing through the outer layer; an electrostatic filter layer for secondarily filtering the first substances harmful to the human body and second substances harmful to the human body; an inner layer for guiding the air secondarily filtered through the electrostatic filter layer to the respiratory system; and a medicine kit layer formed on one side of the inner layer and having a structure capable of accommodating a treatment medicine permeating the human body therein to thus treat diseases, obesity, and health problems.

According to the present invention, the protruding member may include: an outer layer for blocking relatively large particles and water; an anti-bacterial filter layer for primarily filtering first substances harmful to the human body contained in the air passing through the outer layer; an electrostatic filter layer for secondarily filtering the first substances harmful to the human body and second substances harmful to the human body; an inner layer for guiding the air secondarily filtered through the electrostatic filter layer to the respiratory system; a detection kit layer formed on one side of the inner layer to detect diseases, degrees of obesity, and health conditions of the human body, based on substances discharged from the respiratory system; and a medicine kit layer formed on one side of the detection kit layer and having a structure capable of accommodating a treatment medicine permeating the human body therein to thus treat diseases, obesity, and health problems.

According to the present invention, the protruding member may be detachably attached freely to the band part.

According to the present invention, the through hole may have the shape of a rectangle.

According to the present invention, the through hole may have the shape of a circle or oval.

According to the present invention, the through hole may be constituted of a plurality of through holes spaced apart from one another by a given distance on the region facing the respiratory system.

According to the present invention, the band part may have the shape of a patch made of any one selected from synthetic resin, plastic, synthetic fibers, natural fibers, tape, rubber, foams, paper, and a silicone adhesive or a combination of at least two or more materials.

According to the present invention, the band part may be made of a flexible material.

According to the present invention, the band part may have a point adhesion member applied to one side surface thereof.

According to the present invention, the point adhesion member may be made of any one of synthetic resin-based, synthetic rubber-based, nature-based, and acrylic-based point adhesion components, and an eco-friendly point adhesion component or a combination of at least two or more components.

According to the present invention, the band part may include the through hole with various shapes such as rectangle, circle, and oval formed at the given position thereof to introduce the large amount of air into the respiratory system of the human body, while being detachably attached freely to the skin of the human body to surround the region around the respiratory system of the human body through the point adhesion member, and further, the band part may have a plurality of attachment strengthening holes spaced apart from each other by a given distance along the outer edges thereof.

### Advantageous Effects

According to the present invention, the healthcare band for a human body can selectively cover only the mouth or nose of the human body, not surrounding a wide range of all of the mouth (oral cavity) and the nose (nasal cavity) of the human body, thereby improving the wearing comfort of a wearer and enabling wearing for long hours while he or she has a meeting or is talking.

According to the present invention, the healthcare band for a human body can selectively cover only the mouth or nose of the human body, thereby preventing a wearer's glasses from fogging due to the moisture discharged from his or her breathing and suppressing difficulties in breathing, occurrence of headache, and deterioration of health conditions from being caused by the re-suction of carbon monoxide emitted from the respiratory system of the wearer to the wearer's body through his or her respiratory system.

According to the present invention, the healthcare band for a human body can selectively cover only the mouth or nose of the carbon monoxide to thus prevent difficulties in breathing and occurrence of headache while a wearer is taking outdoor exercise, thereby keeping the exercise, improving the wearing comfort of the wearer, and providing a low price healthcare band.

According to the present invention, the healthcare band for a human body can selectively cover only the mouth or nose of the human body, so that it can be used as a disposable product at a place needed.

According to the present invention, the healthcare band for a human body can selectively cover only the mouth or nose of the human body, so that as it is relatively small in volume, it is easy to be carried in a wearer's wallet, backpack, or pocket, as items necessary for his or her trip.

According to the present invention, the healthcare band for a human body can selectively cover only the mouth or nose of the human body, so that it can be used even in hot regions or in summers.

According to the present invention, the healthcare band for a human body can selectively cover only the mouth or nose of the human body through the point adhesion member, so that there is no gap, thereby preventing the harmful substances and fine substances from entering the respiratory system of the human body.

According to the present invention, the healthcare band for a human body can selectively cover only the mouth or nose of the human body, so that various medicines capable of treating diseases such as stroke, cardiovascular diseases, and the like directly permeate the respiratory system of the human body quickly, thereby providing real-time medicinal effects.

According to the present invention, the healthcare band for a human body can selectively cover only the mouth or nose of the human body, thereby preventing all kinds of carcinogens and fine substances harmful to the human body that are generated when a wearer cooks with gas or fire from being sucked to his or her respiratory system to suppress various diseases such as cancers, stroke, heart diseases, and the like from occurring.

According to the present invention, the healthcare band for a human body can selectively cover only the mouth or nose of the human body, while being provided with the sensor module adapted to in real time monitor and store biological signals including a temperature, humidity, sweat, and the like through a mobile application, based on substances discharged from the respiratory system, and to transmit the monitored result to the external receiver, thereby capable of detecting various diseases, degrees of obesity, and health conditions.

### BRIEF DESCRIPTION OF THE DRAWINGS

FIG. 1 is a front view showing a healthcare band for a human body according to an embodiment of the present invention.

FIG. 2 is a front view showing a healthcare band for a human body according to anther embodiment of the present invention.

FIG. 3 is a front view showing a healthcare band for a human body according to yet another embodiment of the present invention.

FIG. 4 is a sectional view taken along the line A-A of FIG. 1.

FIGS. 5(a), 5(b) 5(c), and 5(d) are sectional views showing protruding members selectively applied to the healthcare band for a human body according to the present invention.

### DETAILED DESCRIPTION

Objects, characteristics and advantages of the present invention will be more clearly understood from the detailed description as will be described below and the attached drawings. Before the present invention is disclosed and described, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure.

If it is determined that the detailed explanation on the well known technology related to the present invention makes the scope of the present invention not clear, the explanation will be avoided for the brevity of the description. Further, the terms as will be discussed later are defined in accordance with the functions of the present invention, but may be varied under the intention or regulation of a user or operator. Therefore, they should be defined on the basis of the whole scope of the present invention.

Hereinafter, the present invention will be disclosed in detail with reference to the attached drawings.

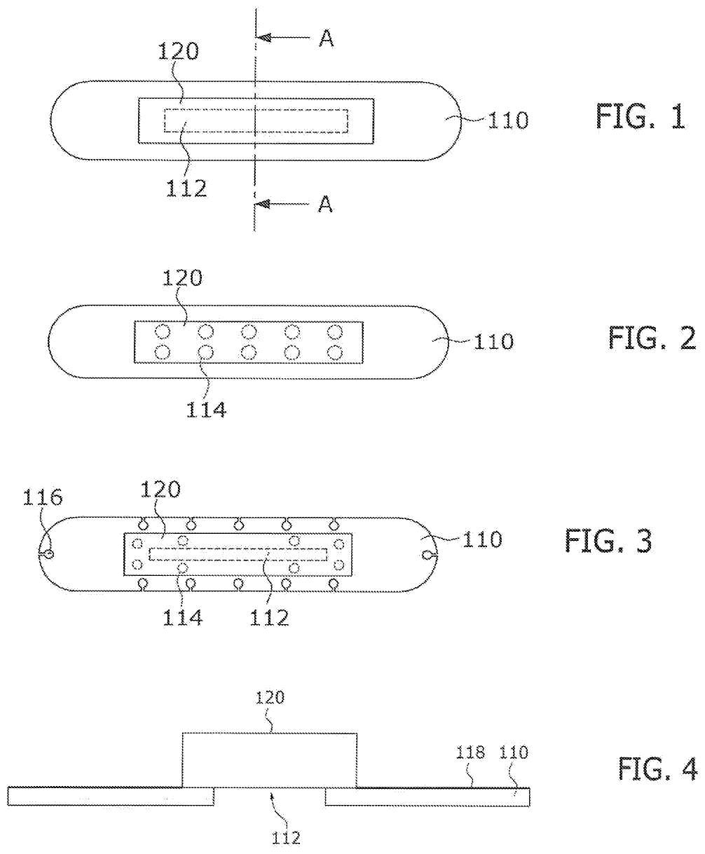

FIG. 1 is a front view showing a healthcare band for a human body according to an embodiment of the present invention, FIG. 2 is a front view showing a healthcare band for a human body according to anther embodiment of the present invention, and FIG. 3 is a front view showing a healthcare band for a human body according to yet another embodiment of the present invention.

Referring to FIG. 1, a healthcare band for a human body according to an embodiment of the present invention largely includes a band part 110 and a protruding member 120.

First, the band part 110 has a through hole 112 formed at a predetermined position (for example, at a roughly center portion) thereof to introduce a large amount of air into the respiratory system of the human body and is detachably attached freely to the skin of the human body to surround the region around the respiratory system through a point adhesion member (See a reference numeral 118 of FIG. 4). In this case, the respiratory system includes the mouth (oral cavity) or nose (nasal cavity) of the human body, and the corners of the through hole 112 may be rounded so that the through hole 112 is kept to a given strength capable of being resistant well to artificial tearing.

Further, the through hole 112, which is formed at the roughly center portion of the band part 110, has various shapes such as a rectangle, a circle, an oval, and the like.

In addition, as shown in FIG. 2, the through hole 112 of the band part 110 is constituted of a plurality of through holes 114 spaced apart from one another by a given distance on the region facing the respiratory system (oral or nasal cavity) of the human body.

Otherwise, as shown in FIG. 3, the through hole 112 of the band part 110 is constituted of a rectangular through hole formed at a center portion of the band part 110 and a plurality of through holes 114 spaced apart from one another by a given distance along the outside edges of the rectangular through hole.

The band part 110 has the shape of a patch made of any one selected from synthetic resin, plastic, synthetic fibers, natural fibers, tape, rubber, foams, paper, and a silicone adhesive or a combination of at least two or more materials. To allow the band part 110 to be attached well to the muscle or skin around the mouth or nose of the human body, the band part 110 is made of any one selected from a synthetic resin adhesive, a synthetic rubber adhesive, a cotton-like natural adhesive, an acrylic adhesive, and the like and a combination of at least two or more adhesives. When considering that the muscle or skin around the mouth or nose of the human body moves, while a wearer is talking or breathing, the band part 110 is made of a flexible material so that it can be resistant to such movements (that is, it can be kept attached well to the wearer's skin).

Moreover, the band part 110 includes a plurality of attachment strengthening holes 116 spaced apart from each other by a given distance along the outer edges thereof so that even if the muscle or skin around the mouth or nose of the wearer moves, while he or she is talking or breathing, the healthcare band can be attached well even to the curved portions such as the mouth, the nose, and the like, while preventing the attachment force from being lowered. In this case, the corners of the attachment strengthening holes 116 may be rounded such that the attachment strengthening holes 116 are kept to a given strength capable of being resistant well to artificial tearing.

Referring back to FIGS. 1 to 3, the protruding member 120 is formed (detachably attached) to surround (or cover) a region around the through hole 112 or the plurality of through holes 114, so that the protruding member 120 prevents substances harmful to the human body contained in the air introduced through the through hole 112 or the plurality of through holes 114 from flowing into the respiratory system of the wearer.

FIG. 4 is a sectional view taken along the line A-A of FIG. 1.

Referring to FIG. 4, the point adhesion member 118 is formed on one side (coming into contact with the skin of the human body) of the band part 110 to perform the point adhesion to the skin, and for example, the point adhesion member 118 is made of a point adhesion component (such as synthetic resin-based, synthetic rubber-based, nature-based, and acrylic-based point adhesion components, a point adhesion component used in a band-aid, and the like) giving no harm to the skin of the human body.

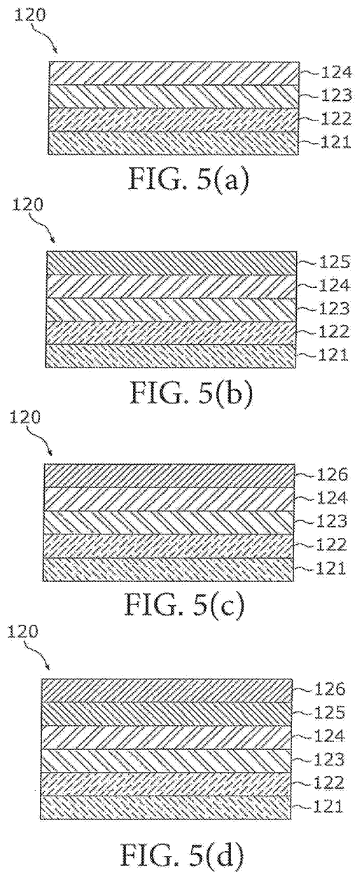

Further, the protruding member 120 is detachably attached (through point adhesion) freely to a given position of the band part 110, while covering the through hole 112, and for example, as shown in FIGS. 5(a), 5(b), 5(c), and 5(d), the protruding member 120 may have a laminate structure capable of selectively performing various functions.

FIGS. 5(a), 5(b), 5(c), and 5(d) are sectional views showing the protruding members selectively applied to the healthcare band for a human body according to the present invention.

Referring to FIG. 5(a), the protruding member 120, which is formed at a given position of the band part 110, while covering the through hole 112 and surrounding the region around the through hole 112, according to an example of the present invention, has a laminate structure of an outer layer 121, an anti-bacterial filter layer 122, an electrostatic filter layer 123, and an inner layer 124.

In this case, the outer layer 121 functions to block relatively large particles and water (for example, fine droplets of water or saliva discharged from the respiratory systems of people around the wearer) from being introduced into the respiratory system (the oral or nasal cavity) of the wearer when he or she inhales air.

Further, the anti-bacterial filter layer 122 functions to primarily filter first substances harmful to the human body contained in the air passing through the outer layer 121.

In this case, the first substances harmful to the human body include viruses, bacteria, carcinogens, microorganisms, pollutants, and the like, and as they are introduced into the human body through the respiratory system, they may cause various diseases such as colds, pneumonia, cancers, respiratory diseases, and the like.

Through the anti-bacterial filter layer 122 of the protruding member 120, accordingly, the first substances harmful to the human body contained in the outside air introduced into the respiratory system can be primarily filtered (blocked).

Further, the electrostatic filter layer 123 functions to filter the first substances harmful to the human body and second substances harmful to the human body, and in this case, the second substances harmful to the human body include yellow sand, fine dust, ultra fine dust, fine substances, and the like. If the first and second substances harmful to the human body are introduced into the human body through the respiratory system, they may cause various diseases such as asthma, dementia, stroke, cardiovascular diseases, heart diseases, and the like.

Through the electrostatic filter layer 123 of the protruding member 120, accordingly, the first and second substances harmful to the human body contained in the outside air introduced into the respiratory system can be secondarily filtered (blocked).

Next, the inner layer 124 functions to guide the air secondarily filtered through the electrostatic filter layer 123 to the respiratory system, and in this case, the inner layer 124 may have a structure in which a medicine (for improving health conditions or treating diseases and obesity) is accommodated so that the medicine is directly sucked quickly to the respiratory system.

Referring to FIG. 5(b), the protruding member 120, which is formed at a given position of the band part 110, while covering the through hole 112 and surrounding the region around the through hole 112, according to another example of the present invention, has a laminate structure of an outer layer 121, an anti-bacterial filter layer 122, an electrostatic filter layer 123, an inner layer 124, and a detection kit layer 125.

Referring to FIG. 5(b), that is, the protruding member 120, which is formed at the given position of the band part 110, while covering the through hole 112 and surrounding the region around the through hole 112, has a laminate structure of the outer layer 121, the anti-bacterial filter layer 122, the electrostatic filter layer 123, the inner layer 124, and the detection kit layer 125, and in this case, the respective layers may be laminated on top of each other by means of point adhesion, melting, and the like.

According to another example of the present invention, the laminate structure of the outer layer 121, the anti-bacterial filter layer 122, the electrostatic filter layer 123, and the inner layer 124 is the same as of FIG. 5(a), and further, the functions of the respective layers are substantially the same as of FIG. 5(a).

However, the protruding member 120 as shown in FIG. 5(b) is different from that as shown in FIG. 5(a) in that the detection kit layer 125 having a given thickness is laid on top of the inner layer 124.

In this case, the detection kit layer 125 is formed (attached through point adhesion or melting) on one side of the inner layer 124 and has a sensor module (not shown) adapted to in real time monitor and store biological signals including a temperature, humidity, sweat, bacteria, viruses, carcinogens, and the like through a mobile application, based on substances (secretion such as saliva, phlegm, runny nose, and the like) discharged from the respiratory system (oral or nasal cavity) and to transmit the monitored result to an external receiver (for example, a smartphone), thereby detecting various diseases such as cancers, degrees of obesity, and health conditions.

If various diseases are detected through the substances discharged from the respiratory system, further, the detection kit layer 125 visually indicates the presence or absence of diseases, for example, through color changing.

Accordingly, the protruding member 120 as shown in FIG. 5(b) further has a function of detecting the diseases when compared with that as shown in FIG. 5(a).

Referring to FIG. 5(c), the protruding member 120, which is formed at a given position of the band part 110, while covering the through hole 112 and surrounding the region around the through hole 112, according to yet another example of the present invention, has a laminate structure of an outer layer 121, an anti-bacterial filter layer 122, an electrostatic filter layer 123, an inner layer 124, and a medicine kit layer 126.

According to yet another example of the present invention, the laminate structure of the outer layer 121, the anti-bacterial filter layer 122, the electrostatic filter layer 123, and the inner layer 124 is the same as of FIG. 5(a), and further, the functions of the respective layers are substantially the same as of FIG. 5(a).

However, the protruding member as shown in FIG. 5(c) is different from that as shown in FIG. 5(a) in that the medicine kit layer 126 having a given thickness is laid on top of the inner layer 124.

In this case, the medicine kit layer 126 is formed (attached through point adhesion or melting) on one side of the inner layer 124 and has a structure capable of accommodating a treatment medicine permeating the human body therein to thus treat various diseases like cancers and obesity or to improve health conditions.

Accordingly, the protruding member 120 as shown in FIG. 5(c) further has a function of providing the treatment medicine needed for treating diseases when compared with that as shown in FIG. 5(a).

Referring to FIG. 5(d), the protruding member 120, which is formed at a given position of the band part 110, while covering the through hole 112 and surrounding the region around the through hole 112, according to still another example of the present invention, has a laminate structure of an outer layer 121, an anti-bacterial filter layer 122, an electrostatic filter layer 123, an inner layer 124, a detection kit layer 125, and a medicine kit layer 126.

According to yet another example of the present invention, the laminate structure of the outer layer 121, the anti-bacterial filter layer 122, the electrostatic filter layer 123, and the inner layer 124 is the same as of FIG. 5(a), and further, the functions of the respective layers are substantially the same as of FIG. 5(a).

However, the protruding member as shown in FIG. 5(d) is different from that as shown in FIG. 5(a) in that the detection kit layer 125 having a given thickness as shown in FIG. 5(b) and the medicine kit layer 126 having a given thickness as shown in FIG. 5(c) are laid sequentially on top of the inner layer 124.

In this case, the detection kit layer 125 and the medicine kit layer 126 have the same functions as shown in FIGS. 5(b) and 5 (c).

Accordingly, the protruding member 120 as shown in FIG. 5(d) further has two functions of detecting the diseases and providing the treatment medicines needed for treating the diseases when compared with that as shown in FIG. 5(a).

To provide various functions, further, the healthcare band for a human body according to the present invention may be configured to have various protruding members with different laminate structures laid on top of each other, to change the order of the lamination of the protruding members, or to allow only some of the protruding members to be laminated on top of each other, such that the healthcare band can be used freely as the harmful substance-blocking band, the detection band for detecting diseases, degrees of obesity, and health conditions, and the treatment band for treating diseases and obesity. Moreover, the healthcare band for a human body according to the present invention may be configured to allow the protruding members to be made and distributed (sold) to the form of being detachably attached freely to the band part, such that the protruding members can be selected appropriately according to a user's desired situation and then attached to the band part. Through such active user selection, accordingly, the healthcare band for a human body according to the present invention can effectively satisfy various service desires of product users.

In addition, the healthcare band for a human body according to the present invention may be attached to the inside of a mask made of cotton, non-woven fabric, and the like, if necessary, so that through the wearing of the mask to which the healthcare band is attached, a variety of selection of the user can be obtained.

As mentioned above, the embodiments of the present invention have been disclosed in the specification and drawings. In the description of the present invention, the embodiments are used not to limit the present invention and the scope of the present invention as defined in claims, but just to explain the present invention. Therefore, persons skilled in the relevant art can appreciate that many modifications and variations are possible in light of the above teachings.

It is therefore intended that the scope of the invention be limited not by this detailed description, but rather by the claims appended hereto.

1. A healthcare band for a human body, comprising:
a band part detachably attached freely to skin of the human body to surround a region around a respiratory system of the human body through a point adhesion member formed on one side of the band part and having a through hole formed at a predetermined position of the band part to introduce air into the respiratory system of the human body; and
a protruding member configured to block the through hole, while surrounding a region around the through hole, to prevent substances harmful to the human body contained in the air introduced through the through hole from flowing into the respiratory system.

2. The healthcare band according to claim 1, wherein the protruding member comprises:
an outer layer for blocking relatively large particles and water;
an anti-bacterial filter layer for primarily filtering first substances harmful to the human body contained in the air passing through the outer layer;
an electrostatic filter layer for secondarily filtering the first substances harmful to the human body and second substances harmful to the human body; and
an inner layer for guiding the air secondarily filtered through the electrostatic filter layer to the respiratory system.

3. The healthcare band according to claim 1, wherein the protruding member comprises:
an outer layer for blocking relatively large particles and water;
an anti-bacterial filter layer for primarily filtering first substances harmful to the human body contained in the air passing through the outer layer;
an electrostatic filter layer for secondarily filtering the first substances harmful to the human body and second substances harmful to the human body;
an inner layer for guiding the air secondarily filtered through the electrostatic filter layer to the respiratory system; and
a detection kit layer formed on one side of the inner layer to detect at least one of disease, degree of obesity, and health condition of the human body, based on substances discharged from the respiratory system.

4. The healthcare band according to claim 1, wherein the protruding member comprises:
an outer layer for blocking relatively large particles and water;
an anti-bacterial filter layer for primarily filtering first substances harmful to the human body contained in the air passing through the outer layer;
an electrostatic filter layer for secondarily filtering the first substances harmful to the human body and second substances harmful to the human body;
an inner layer for guiding the air secondarily filtered through the electrostatic filter layer to the respiratory system; and
a medicine kit layer formed on one side of the inner layer and having a structure capable of accommodating a treatment medicine permeating the human body therein to treat diseases, obesity, and health problems.

5. The healthcare band according to claim 1, wherein the protruding member comprises:
an outer layer for blocking relatively large particles and water;
an anti-bacterial filter layer for primarily filtering first substances harmful to the human body contained in the air passing through the outer layer;
an electrostatic filter layer for secondarily filtering the first substances harmful to the human body and second substances harmful to the human body;
an inner layer for guiding the air secondarily filtered through the electrostatic filter layer to the respiratory system;
a detection kit layer formed on one side of the inner layer to detect diseases, degrees of obesity, and health conditions of the human body, based on substances discharged from the respiratory system; and
a medicine kit layer formed on one side of the detection kit layer and having a structure capable of accommodating a treatment medicine permeating the human body therein to thus treat diseases, obesity, and health problems.

6. The healthcare band according to claim 1, wherein the band part has a shape of a patch made of at least one material selected from materials including synthetic resin, plastic, synthetic fibers, natural fibers, tape, rubber, foams, paper, and a silicone adhesive.

7. The healthcare band according to claim 1, wherein the band part comprises a plurality of attachment strengthening holes spaced apart from each other by a given distance along outer edges thereof.
